(19) United States
(12) Patent Application Publication
(10) Pub. No.: US 2025/0064672 A1
(43) Pub. Date: Feb. 27, 2025

(54) VIBRATING MASTURBATOR DEVICE WITH STERILIZATION AND CHARGING FUNCTIONS

(71) Applicant: California Exotic Novelties LLC, Ontario, CA (US)

(72) Inventor: Chih-Yao Tai, Dongguan City (CN)

(21) Appl. No.: 18/824,337

(22) Filed: Sep. 4, 2024

Related U.S. Application Data
(62) Division of application No. 17/301,973, filed on Apr. 20, 2021, now Pat. No. 12,109,164.

(30) Foreign Application Priority Data
Sep. 24, 2020 (CN) 202022115979.0

(51) Int. Cl. A61H 19/00; A61L 2/10; H02J 7/00

(52) U.S. Cl. CPC A61H 19/34; A61L 2/10; H02J 7/0042; A61L 2202/11; A61L 2202/14

(57) ABSTRACT

A vibrating masturbator device for use with a masturbator device charging box with sterilization and charging functions. In an embodiment, the vibrating masturbator device includes a body having a defined shape with a base end and a vibrating end. The body is tapered toward the vibrating end so as to be insertable, vibrating end first, into a storage compartment of corresponding shape in the masturbator device charging box. A switch control member is disposed on the body. A pair of charging contacts are exposed on the body in order to engage counterpart charging contacts disposed in the masturbator device charging box when the body is inserted in the storage compartment of the masturbator device charging box. A masturbator device battery is electrically connected to the charging contacts. A vibration motor is powered by the battery.

### BACKGROUND

### 1. Field

The present disclosure relates to the technical field of health products, and in particular to vibrating masturbator devices and the sterilization and charging thereof.

### 2. Description of the Prior Art

With the continuous and rapid development of society, people are pursuing both material and spiritual demands. Currently, most people have a well-off life, and enjoy the happiness brought by rich materials. When the material demands reach a peak, people will seek the spiritual demands. However, the pressure brought by working and society makes people suffer stress physically and spiritually, which makes people rarely talk with each other, and even affects the lives of husband and wife, causing disharmony between them.

At present, most females would use masturbator devices to meet their mental demands. Some existing masturbator devices are too big to be taken about, and are not disinfected after using, which could easily cause infection when they are used again. Therefore, there is a need to solve the technical problems that exist currently.

### SUMMARY

The technical problems regarding the current technical disadvantages as above described are solved by a vibrating masturbator device product with sterilization and charging functions. The vibrating masturbator device product includes a vibrating masturbator device and a charging box for placing and charging the masturbator device.

In embodiment, the masturbator device includes a body, a switch control member on the body, charging contacts exposed on the body, a masturbator device battery electrically connected to the charging contacts, and a vibration motor powered by the battery.

In an embodiment, the masturbator device charging box includes a storage compartment for receiving the masturbator device, charging contacts corresponding in position to the charging contacts on the masturbator device, a charging box power source electrically connected to the charging box charging contacts, and a sterilization device arranged to sterilize the masturbator device while it is being charged in the charging box, the sterilization device being powered by the charging box power source.

In an embodiment, the masturbator device includes an outer shell, a shell and a switch control button located on an upper end of the shell. A joint between the outer shell and the shell is provided with a ring-shaped charging sheet. The shell is provided with a first magnetic device, a masturbator device battery, a vibration motor and a switch control plate. The first magnetic device is provided on both sides of the shell, and the masturbator device battery is connected electrically to the ring-shaped charging sheet.

In an embodiment, the masturbator device charging box includes a charging box housing and a storage compartment inside the charging box housing, a charging box battery, a sterilization device, a second magnetic device and a power board. The sterilization device is in a bottom of the storage compartment, and the power board includes a power indicator located at a front of the storage compartment. The second magnetic device is fixed on both sides of the storage compartment and corresponds to the position of the first magnetic device on the masturbator device. The sterilization device and the charging box battery are electrically connected with the power board.

In an embodiment, the masturbator device charging box has a flip-open lid, and an upper inner end of the charging box has a protective gasket.

In an embodiment, the sterilization device in the masturbator device charging box is composed of multiple UVC ultraviolet disinfection lamps.

In an embodiment, the storage compartment in the masturbator device charging box has charging contacts corresponding to the position of the ring-shaped charging sheet on the masturbator device.

In an embodiment, the first magnetic device in the masturbator device and the second magnetic device in the charging box are mutually attracted.

In an embodiment, the masturbator device charging box has a charging port for charging the battery in the charging box.

In an embodiment, the outer shell of the masturbator device is connected and fixed with the shell of the masturbator device by a fastener.

### BRIEF DESCRIPTION OF THE DRAWINGS

The foregoing and other features and advantages will be apparent from the following more particular description of example embodiments, as illustrated in the accompanying Drawings.

FIG. 1 is an overall structure schematic diagram showing an embodiment of the vibrating masturbator device product disclosed herein.

FIG. 2 is an exploded structure schematic diagram showing an embodiment of the masturbator device disclosed herein.

FIG. 3 is an exploded structure schematic diagram showing an embodiment of the masturbator device charging box disclosed herein.

### DETAILED DESCRIPTION OF EXAMPLE EMBODIMENTS

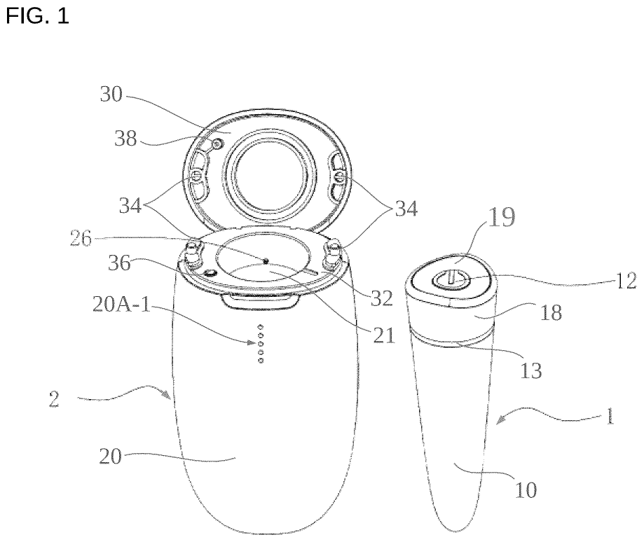

Turning now to the Drawing Figures, which are not necessarily to scale, FIGS. 1-3 illustrate an example embodiment of a vibrating masturbator device product with sterilization and charging functions. As shown in FIG. 1, the vibrating masturbator device product according to the illustrated embodiment includes a vibrating masturbator device 1 and a charging box 2 for placing and charging the masturbator device 1.

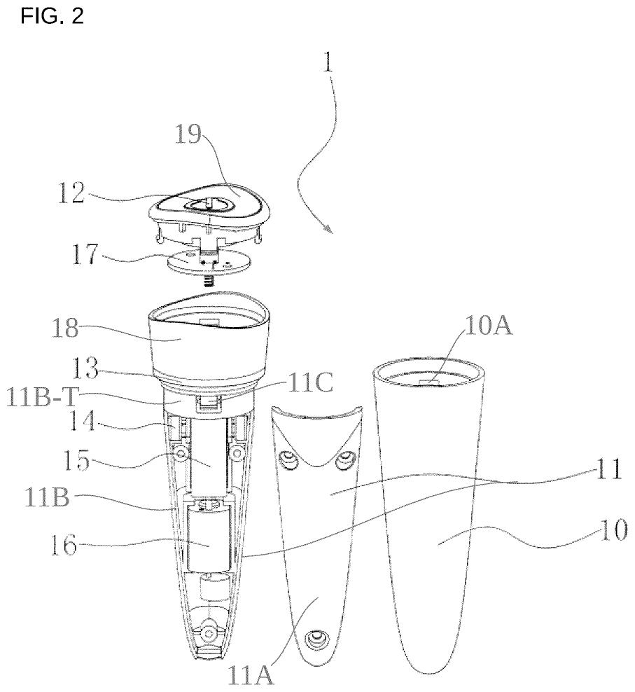

As shown in FIG. 2, the vibrating masturbator device 1 according to the illustrated embodiment includes an outer shell 10 and a shell 11 that define a masturbator device body. The shell 11 includes a front shell half 11A and a rear shell half 11B. The outer shell 10 is connected and fixed with the shell 11 by one or more fasteners, such as a pair of front and rear spring clips 11C on the shell (only the front clip is shown) that engage front and rear spring clip retainers 10A (only the rear retainer is shown) on the outer shell.

The outer shell 10 and a lower portion of the shell 11 define a lower vibrating end of the masturbator body, and of the masturbator device 1 itself. An upper base end of the masturbator body, and of the masturbator device 1 itself, may be provided by a cowling 18 that attaches to a tubular upper end 11B-T of the rear shell half 11B. The cowling 18, which forms an upper extension of the shell 11, serves as a bezel that retains an upper end cover 19 of the masturbator device 1. The upper end cover 19, which may be transparent, forms an upper end of the shell 11. The upper end cover 19 mounts a switch control button 12.

The annular joint between the outer shell 10 and the shell 11 is provided with a ring-shaped charging sheet 13. The ring-shaped charging sheet 13 is formed as a two-piece ring having front and rear ring halves (only the front ring half is shown) that define masturbator device charging contacts. The masturbator device charging contacts are exposed laterally on the masturbator device 1, proximate to its upper base end.

The shell 11 is additionally provided with a first magnetic device 14, a masturbator device battery 15, a vibration motor 16, and a switch control plate 17. With the exception of the switch control plate 17, the foregoing components are carried by the rear shell half 11B, with the front shell half 11A serving as a removable cover. The switch control plate 17 is situated below the upper end cover 12, and is carried by the cowling 18. The first magnetic device 14 is provided on both sides of the shell 11. The masturbator device battery 15 is connected electrically to the ring-shaped charging sheet 13. The switch control button 12 is connected to the switch control plate 17, and controls the operation of the vibrator motor 16.

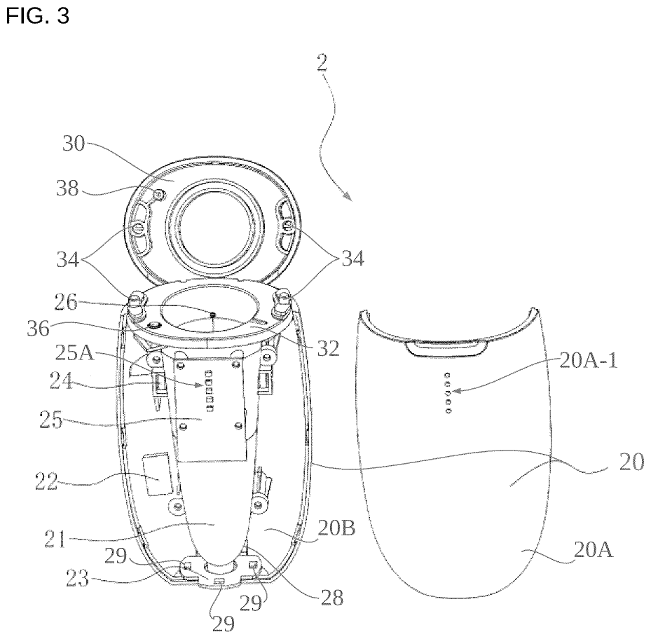

As shown in FIG. 3, the masturbator device charging box 2 according to the illustrated embodiment includes a charging box housing 20 and a storage compartment 21 inside the charging box housing 20. The masturbator device charging box 2 further includes a charging box battery 22, a sterilization device 23, a second magnetic device 24, and a power board 25. The charging box housing 20 includes a front housing half 20A and a rear housing half 20B. The storage compartment 21, the charging box battery 22, the sterilization device 23, the second magnetic device 24, and the power board may all be carried by the rear housing half 20B, with the front housing half 20A serving as a removable cover.

The storage compartment 21 conforms to the shape of the masturbator device body, and particularly the lateral surface contour of the outer shell 10 and the cowling 18 of the masturbator device 1. The sterilization device 23 may be located at the bottom of the storage compartment 21. Thus, the masturbator device 1 can be put into the storage compartment 21 to perform disinfection. The power board 25 includes a power indicator 25A (e.g. an array of indicator lights) located at the front of the storage compartment 21. The power indicator 25A is visible through a power indicator window 20A-1 (e.g., an array of openings) formed in the front housing half 20A. The second magnetic device 24 is fixed on both sides of the storage compartment 21 and corresponds to the position of the first magnetic device 14 on the masturbator device 1. The first magnetic device 14 in the masturbator device 1 and the second magnetic device 24 in the charging box 2 are mutually attracted. The first magnetic device 14 and the second magnetic device 24 may thus both be magnets that work through the effect of magnet attraction. The sterilization device 23 and the charging box battery 22 are electrically connected with the power board 25.

The storage compartment 21 has front and rear charging contacts 26 (only the rear contact is shown) corresponding to the position of the front and rear ring halves of the ring-shaped charging sheet 13 on the masturbator device 1. The masturbator device 1 can receive charging current through the charging contacts 26.

The masturbator device charging box 2 also has a charging port 28 for charging the battery 22 in the charging box.

The sterilization device 23 may be composed of multiple (e.g. 3-4) UVC ultraviolet disinfection lamps 29, which can provide effective sterilization of the masturbator device 1. The UVC ultraviolet disinfection lamps may be located outside (e.g., below) the charging box storage compartment 21, which may comprise UVC ultraviolet light transmissible material, in order to illuminate the masturbator device 1 with UVC ultraviolet light when it is disposed inside of the charging box storage compartment.

The masturbator device charging box 2 may be provided with a flip-open lid 30, and the upper inner end of the charging box 2 may have a protective gasket 32, which protects the masturbator device 1 from being scratched by the flip-open lid. The flip-open lid 30 may be secured in the closed position by friction latches 34. The friction latches 34 include inter-engaging friction latch components respectively formed on the inside of the flip-open lid 30 and on the upper inner end of the charging box 2. The friction latches 34 prevent inadvertent opening of the flip-open lid 30 (and emission of UVC ultraviolet light from the storage compartment 21) while the sterilization device 23 is operating. For additional safety, a switch actuator button 36 may be provided on the upper inner end of the charging box 2. The switch actuator button 36 is engaged by a switch button actuating pin 38 on the flip-open lid 30 when the lid is closed. The switch actuator button 36 actuates a switch that may be electrically connected so that the sterilization device 23 will only activate when the the flip-open lid 30 is closed, and will deactivate when the flip-open lid is opened. The switch actuator button 36 may similarly be used to activate and deactivate the charging function of the masturbator device charging box 2.

Benefits of the above-described vibrating masturbator device product with sterilization and charging functions include: small size, ease of use, and portability. The masturbator device charging box 2 can support the masturbator device 1 for long-term usage with charging. In addition, the vibrating masturbator device product incorporates the sterilization device 23 inside the charging box 2, which can provide charging and disinfection at the same time when the masturbator device 1 is put into the masturbator device charging box after use, thus avoiding bodily harm the next time the masturbator device is used. Due to its battery 22, the charging box 2 is portable, and may be used to sterilize and charge the masturbator device 2 either with or without an external power source.

The above embodiments are for the purpose of illustration and not restriction of the disclosed invention. Therefore, all equivalent changes or modifications made according to the subject matter falling within the scope of the claims are included in the scope of this disclosure.

What is claimed is:

1. A vibrating masturbator device for use with a masturbator device charging box with sterilization and charging functions, the vibrating masturbator device comprising:
a body having a defined shape with a base end and a vibrating end;
the body being tapered toward the vibrating end so as to be insertable, vibrating end first, into a storage compartment of corresponding shape in the masturbator device charging box;
a switch control member disposed on the body;
a pair of charging contacts exposed on the body in order to engage counterpart charging contacts disposed in the masturbator device charging box when the body is inserted in the storage compartment of the masturbator device charging box;
a battery electrically connected to the charging contacts; and
a vibration motor powered by the battery.

2. The vibrating masturbator device of claim 1, wherein the switch control member is disposed proximate to the base end of the body.

3. The vibrating masturbator device of claim 1, wherein the switch control member is disposed at the base end of the body.

4. The vibrating masturbator device product of claim 1, wherein the charging contacts are exposed proximate to the base end of the body.

5. The vibrating masturbator device product of claim 1, wherein the charging contacts are exposed laterally on the body proximate to the base end of the body.

6. The vibrating masturbator device product of claim 1, wherein the charging contacts comprise a ring-shaped charging sheet arranged laterally on the body.

7. The vibrating masturbator device product of claim 1, wherein the vibrating masturbator device comprises a magnetic device on the body arranged for magnetic attraction to a counterpart magnetic device in the storage compartment of the masturbator device charging box when the vibrating masturbator device is inserted therein.

8. The vibrating masturbator device of claim 1, wherein the body includes an outer shell and a shell.

9. The vibrating masturbator device of claim 8, wherein the switch control member comprises a button disposed on an upper end of the shell.

10. The vibrating masturbator device of claim 8, wherein the charging contacts comprise a ring-shaped charging sheet arranged laterally proximate to an upper end of the shell.

11. A vibrating masturbator device for use with a masturbator device charging box with sterilization and charging functions, comprising:
a outer shell;
a shell that carriers a battery, a vibration motor and a switch control member;
a switch control button disposed on an upper end of the shell;
an annular joint between the outer shell and the shell; and
a ring-shaped charging sheet disposed at the annular joint.

12. The vibrating masturbator device of claim 11, wherein the shell comprises a front half shell that serves as a removable cover and a rear half shell that carries the battery and the vibration motor.

13. The vibrating masturbator device of claim 11, wherein the outer shell is affixed to the shell by spring clips on the shell that engage spring clip retainers on the outer shell.

14. The vibrating masturbator device of claim 11, wherein the outer shell and a lower portion of the shell define a lower vibrating end of the body, and of the vibrating masturbator device itself.

15. The vibrating masturbator device of claim 11, wherein an upper extension of the shell comprises a cowling that serves as a bezel that retains an upper end cover of the vibrating masturbator device.

16. The vibrating masturbator device of claim 15, wherein the upper end cover mounts the switch control button.

17. The vibrating masturbator device of claim 11, wherein the ring-shaped charging sheet comprises a two-piece ring having front and rear halves that define a pair of charging contacts exposed laterally on the vibrating masturbator device.

18. The vibrating masturbator device of claim 11, wherein the shell is provided with a magnetic device that affixes the vibrating masturbator device to the masturbator device charging box.

19. The vibrating masturbator device of claim 18, wherein the magnetic device is provided on two sides of the shell.

20. A masturbator device for use in a vibrating masturbator device charging box with sterilization and charging functions, comprising:
a body having a defined shape with a base end and a vibrating end;
a switch control member disposed at the base end of the body;
a pair of charging contacts exposed laterally on the body proximate to the base end of the body;
a masturbator device battery electrically connected to the charging contacts; and
a vibration motor powered by the battery.
